(19) United States
(12) Patent Application Publication
(10) Pub. No.: US 2008/0118961 A1
(43) Pub. Date: May 22, 2008

(54) Biodiesel Production From Soapstock

(76) Inventors: Setsuo Sato, Sao Jose dos Campos, SP (BR); Wanderson Bueno De Almeida, Sao Jose dos Campos, SP (BR); Alexssander Shigueru Araujo, Sao Jose dos Campos, SP (BR)

Correspondence Address: COGNIS CORPORATION;PATENT DEPARTMENT, 300 BROOKSIDE AVENUE, AMBLER, PA, 19002, US

(21) Appl. No.: 11/667,390

(22) PCT Filed: Nov. 9, 2004
(86) PCT No.: PCT/BR04/00218

§ 371 Date: Nov. 20, 2007

(51) Int. Cl. C12P 7/64

(52) U.S. Cl. 435/134

(57) ABSTRACT

A process for the production of fatty acid esters from any soapstock generated in the alkali refining process of soybean, sunflower, rice, corn, coconut, palm kernel, rapeseed or cotton oils, which soapstock comprises water, about 0.1-2.0%, fatty derivatives, including glycerides, neutralizing and splitting the soaps with strong acids, followed by enzymatic esterification using a lipase enzyme, with an alkanol, at 15-to-70° C., followed by agitating the resulting mixture for three-to-five days, then separating the crude ester phase from the mixture, neutralizing the residual acid value with a base, and distilling off and recovering the esters, and obtaining a residue containing sterols, wherein the fatty acid esters thus produced may be used as biodiesel fuel.

### CROSS-REFERENCE TO RELATED APPLICATIONS

This application is filed under 35 U.S.C. § 371 claiming priority from Application PCT/BR2004/000218, filed on Nov. 9, 2004, the entire contents of which are incorporated herein by reference.

### FIELD OF THE INVENTION

This invention relates to a process for producing alkyl fatty acid esters, preferably methyl and ethyl fatty acid esters, via enzymatic catalysis, using as feed soapstock waste generated by vegetable oil refineries during the alkali refining process to produce edible oils. The combination of this technology with feedstock availability offers an economic and competitive approach to produce biodiesel or raw material for the chemical industry. Additionally, a new source of sterols is available for the food industry. Converting byproducts from renewable sources into more added-value products using biotechnology is another example of a contribution from the chemical industry using more environmentally-friendly practices.

### BACKGROUND AND RELATED ART

For each metric ton of alkali-refined vegetable oil produced in the world, approximately 30 kg of soapstock is generated. This soapstock represents a high potential source of raw material, since vegetable oil production is growing, especially from the increasing soybean production in Brazil.

Soapstock waste has been used mostly as animal feed, and as a raw material for soap makers, and for fatty acid production. The existent patents and commercial processes to make fatty acids from soapstock employ hydrolysis and acidification steps using strong acids, such as sulfuric or hydrochloridic acids, producing a mixture of fatty acids, inorganic salts, water, and other lesser components, such as glycerin and phospholipides. Due to the nature of this complex mixture, separation of the crude fatty acids layer representing the organic phase from the aqueous phase, is difficult, frequently demanding steps such as water washing, settling out, centrifuging, and filtration to separate the other components from the fatty acids. Some novelty has been introduced lately, for instance, potassium hydroxide has been proposed for use in the caustic refining process, as it generates lower viscosity feedstock, allows a more effective separation of the refined oil from the soapstock, and results in a reduced formation of gum, which otherwise would create significant purification problems, increase costs, and reduce the amount and purity of the desired refined oil, as described in published United States Patent Application 2003/0236422. Also, U.S. Pat. No. 5,156,879 proposes the addition of propionic acid to solubilize the gums present in a soapstock obtained from the alkali refining of a fat, which soapstock has been further pretreated by adding up to about 5% of a mixture of bases then heated to from 100° F. to 300° F.

U.S. Pat. No. 6,475,758 discloses the use of an endogenic bacteria to acidify soapstock by fermentation of endogenous soapstock nutrients and added nutrients, including carbohydrate, nitrogen, phosphorous, and/or sulfur using acidogenic bacteria. This acidulation reaction reportedly avoids the use of strong acids for the treatment of soapstock, which would have necessitated neutralization with a base, thus minimizing wastewater contamination from the resulting salts, and produces potentially valuable by-products, including lactic acid, acetic acid, glyceric acid and nutrient-rich microorganisms that may be a concentrated source of nutrients for animal feed or even human consumption.

The above-mentioned processes result in dark color crude fatty acids having residual moisture and other lesser impurities. Drying and distillation steps are usually necessary in order to produce commercial fatty acids that may be sold in the marketplace or used as esterification feed, because impurities are known to lower esterification reaction speed.

### SUMMARY OF THE INVENTION

The present invention relates to a compact and more environmentally-sound fatty acid esterification production process for making alkyl, mainly methyl and ethyl, esters starting directly with soapstock waste, and using enzymes as esterification catalysts that are able to convert the free fatty acids into esters in the presence of water, salts, soaps, and many other impurities. This esterification step is a benefit of the process and key for the economics of the commercial scale processes, as the esters have much lower viscosities and solidification points than their respective fatty acids, making easier the separation of organic and aqueous phases, which reduced interaction between the phases, in turn, facilitates the purification process, demanding less process steps, as just settling out the mixture is enough to separate the two phases. Once separated by filtration or any other suitable method, the water phase, which is rich in sodium sulfate, may be used as a raw material in the delignification step of the sulfate kraft process in the paper mill industry, and the residue from a distillation step, after neutralization of the fatty acids in the organic phase, representing about 15% by weight of that phase, is rich in sterols, which may be recovered using known processes as shown, for example, in U.S. Pat. No. 6,281,373 B1.

### BRIEF DESCRIPTION OF THE DRAWINGS

FIGS. 1 and 2 show two possible diagrams of the process according to the instant invention.

### DETAILED DESCRIPTION OF THE INVENTION

Other than in the operating examples, or where otherwise indicated, all numbers expressing quantities of ingredients or reaction conditions used herein are to be understood as modified in all instances by the term “about”.

This invention is directed to a process for the production of fatty acid esters directly from any soapstock generated in the alkali refining process, which soapstock contains 10-60% water, 0.1-2.0% of sterols, and 35-85% of fatty derivatives including partial glycerides, by:
neutralizing and splitting the soaps with a strong acid until a pH of from 2-to-8 is reached;
enzymatic esterification using a lipase enzyme in a concentration ranging from 100 ppm-to-10%, by weight, with a C₁-to-C₆ alkanol in a weight ratio of 5-100% in relation to the fatty components, working at a temperature of from 15-70° C. in a batch or continuous process;
agitating the mixture, either mechanically or just by circulation during three-to-five days at a temperature of from 15-70° C. The acid number/acid value is measured in the organic layer during this agitation, with the reaction being stopped when the acid number/acid value has not decreased for 24 hours;
separating the ester phase of the mixture by settling out, by pumping the mixture into a centrifuge, or by filtering out the solids in order to facilitate the separation;
neutralizing the residual acid value from the incomplete enzymatic esterification using an alkali solution selected from the group consisting of sodium, potassium, calcium, aluminum and lithium alkali solutions, ammonium hydroxide, and derivatives thereof, such as organic amines;
distilling off the crude esters using a batch technique or continuous flash distillators, with residual amounts of moisture or methanol being stripped off using a degasser, just prior the main distillation where 80 to 90% of light color esters are produced continuously.

Soapstocks usually have 10-60% of water when they come from alkali neutralization, and most refineries actually add extra water to make the soaps more pumpable, the remaining part of the soapstock being composed of fatty acid soaps themselves, 0.1-2% sterols, some mono-, di- and triglycerides, and also low levels of phospholipides. Some soapstocks likely also contain proteins coming from the extraction process, which proteins would end up as a solid material in the process.

In a preferred embodiment, the invention deals with a process where (a) the soapstocks from alkali refining are selected from the group consisting of soybean, sunflower, rice, corn, coconut, palm kernel, rapeseed and cotton oil soapstocks, (b) the acids used to split the soaps are strong acids, like sulfuric acid or hydrochloridic acids, and (c) the preferred pH is 3.5-6 most preferable 5.

After neutralization, an alkanol, preferred methanol or ethanol, is added to the mixture followed by the specific enzyme. The acid value is measured in the organic layer, which has been separated in a lab centrifuge. Enzymatic catalysis using a liquid lipase, preferably Candida antartica Lipase B, was surprisingly much more effective than running a pure fatty acid esterification, possibly being explained by the fact that some impurities may be acting as surfactants for the system.

Another preferred embodiment involves the use of lipases that have been produced by an organism selected from the group consisting of Aspergillus niger, Aspergillus oryzea, Bacillus species, Candida albicans, Candida antarctica, Candida cylindracea, Candida glabrata, Candida maltosa, Candida parapsilosis, Candida lipolytica, Candida tropicalis, Candida viswanathii, Chromobacterium viscosum, Geotrichum candidum, Issatchenkia orientalis (Candida krusei), Kluyveromyces marxianus (C. kefyr or C. pseudotropicalis), Mucorjavanicus, Penicilium camenberti, Penicilium roqueforti, Pichia guilliermondii (Candida guilliermondii), Porcine pancreas, Pseudomonas cepacia, Pseudomonas fluorescens, Rhizomucor miehei, Rhizopus arrhizus, Rhizopus oryzae, Rhizopus niveus, Rhizopus javanicus, Thermomyces lanugenosus and mixtures thereof, each in the form of a liquid, a solid or immobilized on a substrate material. It is also preferred that the lipase enzyme is a lipase of type B, preferably a Candida antarctica Lipase B. The preferred concentration for the lipase enzymes ranges from 100 ppm-to-10%, and most preferred is 500 ppm.

A further preferred embodiment of the invention relates to the alkanols to be esterified. The preferable alkanol is a linear- or branched-C₁-to-C₆ alkanol, preferred C₁-methanol or C₂-ethanol using batch or continuous technique.

The mixture is agitated mechanically or just by circulation during three-to-five days at temperatures of from 15-to-70° C., preferably at room temperature or from 40-to-60° C., and most preferably at 30-to-45° C. The acid value/acid number is measured in the organic layer during those days, and the reaction is stopped when the acid value has not decreased further for 24 hours. Usually the esterification yield is 80-to-90%, with separation between the organic and aqueous phases becoming easier as esterification increases.

After esterification is complete, the agitation or circulation is stopped, and the ester phase of the mixture is settled out, pumped to a centrifuge, or the solids are filtered out, in order to facilitate the separation.

After complete aqueous phase separation, the residual free fatty acids in the organic phase, from the incomplete esterification, are neutralized with an alkaline solution, with the neutralization water being separated by decanting or by a degassing system during the further distillation step, resulting in light-colored esters.

In a further preferred embodiment of the invention, the distillation step for distilling off the crude esters is carried out using a batch technique or continuous flash distillators, preferably by a thin-film or wiped-film evaporator, with the continuous distillation being operated at 180° C.-240° C. and 1-10 mm Hg pressure, preferable at 220° C. and 3 mm Hg.

Residual amounts of moisture or methanol are stripped off using a degasser just prior to the main distillation still. By this method, 80 to 90% of light color esters are produced continuosly.

The residue, dark in color, coming from the still contains about 5 to 8% of sterols, with the remaining part being fatty material, and both the sterols and the fatty acids are recoverable using the same equipment, using technologies described in the U.S. Pat. No. 6,281,373 B1.

This process will make possible the use of soapstock in more added-value applications, other than conventional animal feed and soap making.

The advantage of the process just described are:
1) the use of enzyme catalysis permits esterification in the presence of a large amount of water and in the presence of other components, other than fatty material;
2) esterification at low temperatures, which simplifies the process and the equipment needed, and permits the use of existent plants, with minor changes. For new plants, capital investment is considerably lower;
3) easy separation of the aqueous phase from the other impurities, which increases dramatically the process yield compared with the conventional fatty acid process. As a consequence, less waste is generated in this process, and the water waste from the process, which is rich in sodium sulfate, may be recovered and used in the sulfate kraft process in the paper mill industry;
4) the availability of sterols, a raw material increasingly in demand around the world, and recovered in this process, which could have a dramatic impact;
5) the simplicity of this process, which would allow small production plants for esters to be located near the vegetable oil refineries, saving a lot of handling and transportation costs; and
6) production of biodiesel for trucks used to transport oils and grains, in addition to production of conventional esters applicable for the chemical industry.

One of the key aspects of this process is the importance of getting the right quality soapstocks. Less water is better for the process, but water is necessary to make soap pumpable. Adding methanol to the pipeline just after esterification and separation out of the solids reduces the viscosity dramatically, meaning that no extra water has to be added. Another approach is to use potassium or lithium hydroxide during refining, which gives lower viscosity for the soapstock.

The ratio, by weight, of soapstock fatty material to C₁ and C₂ alkanol is from 10:2 to 10:0.7, preferably 10:1.5, and the ratio, by weight, of fatty material in the soapstock to enzymes is 10:0.001 to 10:0.200, preferably 10:0.005. The water amount in the soapstock is 10 to 60%, preferably below 40%, and the distillator temperature in the wiped-film evaporator is preferably in the range of 200-225° C., with a pressure of from 1 to 5 Torr.

Another aspect of the invention is the use of fatty acid esters produced according to process of the invention as biodiesel.

The following Examples are intended to exemplify the instant invention, and should not be interpreted as limiting it.

### EXAMPLES

### Example 1.1

100 kg of soapstock with 40% water, a fatty part composed of 95.2% of fatty acids as soaps, 1.2% monoglycerides, 1.5% diglycerides, 1.1% triglycerides, and 1.0% sterols, measured by gel permeation chromatography, was neutralized with 8.7 kg of sulfuric acid, 98% at 45° C. until the pH reached 4.0. 9.0 kg of methanol was added, followed by 0.03 kg of liquid enzyme CALB (Candida antartica Lipase B from Novozymes), and the mixture was kept circulating for six days using a diaphragm pump at 100 liter/hour flow rate. The initial acid number/acid value for the organic phase was 155, and decreased as described in Table 1. External temperature ranged from 24° C. to 30° C. over the six days.

TABLE 1
Acid values for the reaction over six days

| days | AV mgkoh/g |
|---|---|
| 0 | 154 |
| 1 | 105 |
| 2 | 76 |
| 3 | 61 |
| 4 | 48 |
| 5 | 38 |
| 6 | 32 |

### Example 1.2

Circulation was stopped, and the mixture was settled out for eight hours. About 49 kg of crude methyl esters was separated from 68.9 kg of an aqueous phase, including a layer of an emulsion, which was filtered out through a press filter, producing 11 kg of filtration cake. The filtered liquor was settled for an additional three hours, resulting in 2.3 kg of crude methyl esters separated from 55.6 kg of a transparent aqueous phase, which was discharged to the sewer.

The total amount of crude methyl esters produced was 51.3 kg with an acid value of 25. About 1.8 kg of sodium hydroxide in a 50% solution was added to neutralize the residual non-esterified fatty acids.

The total amount of crude methyl esters after neutralization was 53.1 kg for distillation.

### Example 1.3

The crude neutralized methyl esters were fed to a 0.13 ft2 lab wiped-film evaporator at a 1 kg/hour flow rate, with a still temperature of 220° C., at 1.5 mm Hg pressure. A degasser, operating at 150° C. at 5 mm Hg, was assembled before the main still to remove residual water coming from the neutralization and methanol.

40 kg of pure fatty acid methyl esters Gardner 4, with an acid value <2 was produced as a main product, in a yield of 40%, in relation to the soapstock, and 71%, in relation to the total fatty material.

11.1 kg of a bottom stream having 8% sterols, some glycerides, and fatty acid soaps were produced as residue. This material was processed according to U.S. Pat. No. 6,281,373 B1, in order to recover the sterols and the fatty acids as methyl esters again. The bottom stream could also be recycled back to the soapstock storage tank.

### Example 2.1

100 kg of soapstock from the same source as in Example 1.1 was neutralized with 8.7 kg of sulfuric acid, 98% at 45° C., until a pH of 4.0 was reached. 10.0 kg of ethanol 96% was added, followed by 0.03 kg of liquid enzyme CALB (Candida antartica Lipase B from Novozymes). The mixture was kept circulating using a diaphragm pump at 80 liter/hour flow rate for six days. The initial acid number/acid value for the organic phase was 150, and decreased to 38, and the external temperature ranged from 22° C. to 32° C., over the six days.

### Example 2.2

Circulation was stopped and the mixture was settled out for six hours. About 51 kg of crude methyl esters was separated from 68.9 kg of an aqueous phase, including a very small layer of an emulsion, with the aqueous phase being filtered out through a press filter, producing 11 kg of filtration cake. The filtered liquor was settled for an additional three hours, separating 1.3 kg of crude methyl esters from 55.2 kg of a transparent aqueous phase, which was discharged to the sewer. The total amount of crude ethyl esters produced was 52.0 kg, with an acid value of 38. About 3.0 kg of sodium hydroxide in a 50% solution was added to neutralize the residual non-esterified fatty acids.

After neutralization, 55.0 kg of neutralized crude ethyl esters was recovered for distillation.

### Example 2.3

The crude neutralized ethyl esters were fed to a 0.13 ft2 lab wiped-film evaporator at 1 kg/hour flow rate, with a still temperature of 230° C., at 1.0 mm Hg pressure. A degasser, operated at 150° C. at 5 mm Hg, was assembled before the main still to remove residual water coming from the neutralization and ethanol.

42 kg of pure fatty acid methyl esters Gardner 4, with an acid value <2 was produced as a main product, at a yield of 42%, in relation to the soapstock, and 74%, in relation to the total fatty material.

10.0 kg of a bottom stream having 8.4% of sterols, some glycerides, and some fatty acid soaps were produced as residue. This material was processed according to U.S. Pat. No. 6,281,373 B1, in order to recover the sterols. The bottom stream could also be recycled back to the soapstock storage tank.

### Example 3

1 kg of soapstock from the same source as Example 1.1 was neutralized with 0.087 kg of sulfuric acid, 98% at 45° C., until a pH of 4.0 was attained. 0.10 kg of ethanol, 96%, was added, followed by 0.08 kg of Novozym 435 enzyme, adsorbed on a macroporous resin. The mixture was kept under slow mechanical agitation, reaching an acid value of 20, from an initial acid value of 155, after three hours of reaction. The temperature was 35° C. during the esterification time.

### Example 4

1 kg of soapstock from the same source as Example 1.1 was neutralized with 0.087 kg of sulfuric acid, 98% at 45° C., until a pH of 4.0 was reached. 0.09 kg of methanol was added, followed by 0.0005 kg of liquid enzyme CALB (Candida Antartica Lipase B from Novozymes). The mixture was kept under slow mechanical agitation until the acid value reached 18 from the initial 158, after two hours of reaction, with the temperature being 30° C. during the reaction time.

### FIELD OF INVENTION

This invention relates to a process which produces alkyl fatty acid esters and preferable methyl and ethyl fatty acid esters via enzymatic catalysis using as feed soapstock waste generated by the vegetable oil refineries during the alkali refining process to produce edible oils. The combination of this technology with feedstock availability offers an economic and competitive approach to produce biodiesel or raw material for the chemical industry. Additionally a new source of sterols is available for the food industry. Converting byproducts from renewable sources into more added value products using biotechnology is another real case of contribution from the chemical industry using more environmental friendly practices.

### STATE OF THE ART

For each metric ton of alkali refined vegetable produced in the world approximately 30 kg of soapstock is generated. There is a high potential source of raw material since vegetable oil production is growing, specially soybean in Brazil.

Soapstock waste has been used mostly as animal feed, raw material for soap makers, and feed stock for fatty acid production. The existent patents and commercial processes to make fatty acids from soapstock always refers to hydrolysis and acidification steps using strong acids such as sulfuric or hydrochloridic acids, producing a mixture of fatty acids, inorganic salts, water, and other small components such as glycerin, phospholipides. Due to the nature of this complex mixture separation of the crude fatty acids layer representing the organic phase from the aqueous phase is difficult demanding most of the time steps such as water washing, settling out, centrifuging, and filtration to separate the other components from the fatty acids. Some novelty has been introduced lately, for instance, the use of potassium soaps which generates lower viscosity feedstock, one of the biggest problem with sodium soaps, as described in the US patent 20030236422. Another patent disclosing procedure to make fluid soapstock is described in the U.S. Pat. No. 5,156,879. The invention is directed to a method for treatment of soapstock obtained by alkali refining of fats to provide a fluid, uniform, pumpable animal feed product. In the method, a raw soapstock is provided. The soapstock is pretreated by adding a strong, soluble base to the soapstock. Propionic acid is then added to the pretreated soapstock and the pH is adjusted to provide an acidified soapstock. With soapstocks having low gum levels, a fluid, uniform, pumpable product is provided without further treatment. At higher gum levels, the pretreated soapstock and/or the acidified soapstock is heated to a predetermined temperature to provide the fluid, uniform, pumpable product

The U.S. Pat. No. 6,475,758 disclose the use of an endogenic bacteria to acidulate soapstock. It is advantageously acidified by fermentation of endogenous soapstock nutrients and added nutrients under controlled conditions using acidogenic bacteria. The nutrients may include carbohydrate, nitrogen, phosphorous, sulfur from defined or undefined sources. The acidification reaction avoids the use of strong acids for the treatment of soapstock, minimizes wastewater contamination with salts and produces potentially valuable by-products including lactic acid, acetic acid, glyceric acid and nutrient rich microorganisms.

All the above mentioned processes end up with a dark color crude fatty acids having residual moisture and other small components. Drying and distillation steps usually are necessary to produce commercial fatty acids to be sold in the marketplace or to use it as esterification feed because impurities is known to lower esterification reaction speed.

### SUMMARY OF INVENTION

The present invention relates to a compact and more environmentally fatty acid esterification production process to make alkyl and mainly methyl and ethyl esters starting directly from soapstock waste. The benefit of this process is the use of enzymes as esterification catalysts able to convert the free fatty acids into esters in the presence of water, salts, soaps, and many other impurities. This initial step is key for the economics of the commercial scale processes, because the esters has much lower viscosities and solidification point than their respective fatty acids making easier the separation of organic and aqueous phases. The lower interaction between aqueous and organic phases compared to fatty acids also facilitate the purification process. This facile separation makes this step more productive demanding less process steps, just settling out the mixture is enough to separate the two phases. The water phase rich in sodium sulfate separated from the process via filtration or any other suitable method can be used as raw material in the paper mill industry during the delignification step using the sulfate Kraft process. Enzymatic catalyst using a liquid Lipase preferably Candida antartica Lipase B surprisingly was much more effective than running a pure fatty acid esterification. This unbiguous result would be explained by the fact that some impurities may be playing as surfactants for the system. After complete aqueous phase separation the residual free fatty acids in the organic phase is neutralized with alkaline solution, neutralization water is separated by decanting or via degassing system during the further distillation step producing a light colored esters. The residue from the distillation step representing about 15% in weight is rich in sterols which can be recovered using known processes for instance U.S. Pat. No. 6,281,373 B1

### BRIEF DESCRIPTION OF THE DRAWINGS

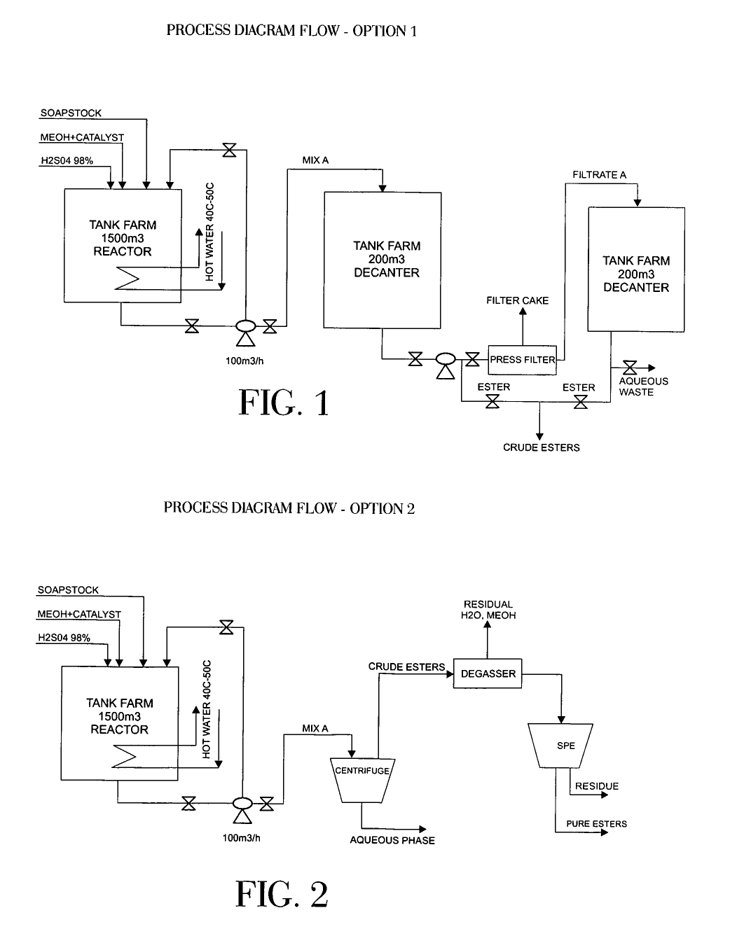

FIGS. 1 and 2 show two possible process diagrams of the claimed process.

### DETAILED DESCRIPTION OF THE INVENTION

Other than in the operating examples, or where otherwise indicated, all numbers expressing quantities of ingredients or reaction conditions used herein are to be understood as modified in all instances by the term “about”.

The subject of the invention is a process for production of fatty acid esters directly from any soapstock generated in the alkali refining process which contains 10-60% water, 0.1-2.0% of sterols, 35-85% of fatty derivatives including partial glycerides, by
neutralizing and splitting the soaps with strong acids until reaching pH 2-8,
followed by enzymatic esterification using Lipase with concentration ranging from 100 ppm-10% by weight, using C1 to C6 alkanol in a weight ratio of 5-100% in relation to the fatty components working at temperature from 15-70° C. via batch or continuous processes,
the mixture is agitated mechanically or just by circulation during three to five days at temperature from 15-70° C. Acid value is measured in the organic layer along the days. Reaction is stopped when acid value does not decrease after 24 hours,
to separate the ester phase the mixture would be settled out, pumped to a centrifuge, or solids is filtered out to facilitate the separation,
the residual acid value from the incomplete enzymatic esterification is neutralized using alkali solution selected from the group consisting of Sodium, Potassium, Calcium, Aluminium, Lithium alkali solutions or ammonium hydroxide and their derivatives such as organic amines,
the crude esters is distilled off using batch technique or continuous flash distillators, residual amount of moisture or methanol is stripped off using a degasser just prior the main distillation still 80 to 90% of light color esters is produced continuously.

Soapstocks usually has 10-60% of water coming from alkali neutralization, and most refineries add extra water to make the soaps pumpable, the remaining part is composed by fatty acid soaps itself, 0.1-2% sterols, presence of mono-, di- and triglycerides and also low level of phospholipides. Some feedstock should also contain proteins coming from the extraction process which would end up as a solid material in the process.

In a preferred embodiment the invention deals with a process where the soapstocks from alkali refining is selected from the group consisting of soybean, sunflower, rice, corn, coconut, palm kernel, rapeseed or cotton and where the acids used to split the soaps are strong acids like sulfuric acid or hydrochloridic acids and the preferred pH is pH 3.5-6 most preferable pH 5.

After neutralization alkanol, preferred methanol or ethanol is added to the mixture followed by the specific enzyme. Acid value is measured in the organic layer separated in a lab centrifuge.

Another preferred embodiment are the possible Lipases to be used in teh process which are produced by an organism selected from the group consisting of Aspergillus niger, Aspergillus oryzea, Bacillus species, Candida albicans, Candida antarctica, Candida cylindracea, Candida glabrata, Candida maltosa, Candida parapsilosis, Candida lipolytica, Candida tropicalis, Candida viswanathii, Chromobacterium viscosum, Geotrichum candidum, Issatchenkia orientalis (Candida krusei), Kluyveromyces marxianus (C. kefyr, C. pseudotropicalis), Mucorjavanicus, Penicilium camenberti, Penicilium roqueforti, Pichia guilliermondii (Candida guilliermondii), Porcine pancreas, Pseudomonas cepacia, Pseudomonas fluorescens, Rhizomucor miehei, Rhizopus arrhizus, Rhizopus oryzae, Rhizopus niveus, Rhizopus javanicus and Thermomyces lanugenosus and mixtures thereof each in the form of liquid, bulk or immobilisied. It is also preferred that the Lipase is a Lipase of type B preferable a Candida antarctica Lipase B. The preferred concentration for the Lipases ranges from 100 ppm to 10% and most preferred is 500 ppm.

A further preferred embodiment of the invention relates to the alkanols to be esterified. The preferable alkanol is a linear or branched C1 to C6 alkanol, preferred C1 Methanol or C2 Ethanol using batch or continuous technique.

The mixture is agitated mechanically or just by circulation during three to five days at temperatures from 15-70° C. preferred at room temperature or preferable at 40-60° C. and most preferable at 30-45° C. Acid value is measured in the organic layer along the days. Reaction is stopped when acid value does not decrease after 24 hours. Usually esterification yield is 80 to 90%. Separation between organic and aqueous phase will become easier as esterification increases.

After esterification gets flat condition, the agitation or circulation is stopped. To separate the ester phase the mixture woul be settled out, pumped to a centrifuge, or solids is filtered out to facilitate the separation.

The residual acid value from the incomplete enzymatic esterification is neutralized using alkali solution.

In a further preferred embodiment of the invention the distillation step is carried out by batch or continuous operation preferable by a thin film or wipped film evaporator and that a continuous distillation is operated at 180° C.-240° C. at 1-10 mmhg pressure, preferable 220° C. at 3 mmhg.

The crude ester is distilled off using batch technique or continuous flash distillators such as thin film or wipped film evaporators. Residual amount of moisture or methanol is stripped off using a degasser just prior the main distillation still. 80 to 90% of light color esters is produced continuously.

The residue coming from the still dark in color has about 5 to 8% of sterols, with the remaining part as fatty material. Sterols and fatty acids can be recovered using the same equipments using technologies described in the patents U.S. Pat. No. 6,281,373 B1

This process will make possible the use of soapstock in more added value applications, other than conventional animal feed and soap makers.

The advantage of the process just described are:
1) the use of enzyme catalysis able to run esterification in the presence of high amount of water and presence of other components other than fatty material
2) esterification at low temperatures simplify process and equipments. This gives this process high flexibility allowing the use of existent plants with minor changes. For new plants capital investment is considerable lower
3) the easy separation of the aqueous phase and other impurities increases dramatically the process yield compared to the fatty acid process. As consequence less waste is generated in this process. The water waste from the process rich in sodium sulfate can be recovered in the sulfate Kraft process used in the paper mill industry.
4) Sterols, a raw material with increased demand nowadays is recovered in this process, this would change dramatically the availability of this raw material around the world.
5) Process simplicity would allow the existence of small esters production plants near to the oil refineries saving a lot of handling and transportation cost
6) Biodiesel for the tank wagons used to transport oils and grains would be produced in that way, in addition to conventional esters application for the chemical industry.

One of the key point of this process is to get the right quality soapstocks. Less water is better for the process, but water is necessary to make soap pumpable. Methanol adding in the pipeline just after the refining centrifuge reduce dramatically the viscosity meaning no need of extra water addition. Another approach is to use potassium or lithium during refining which gives lower viscosity for the soapstock.

The ratio by weight of soapstock fatty material and C1 and C2 alkanol is from 10:2 to 10:0.7 preferably 10:1.5.

The ratio by weight of fatty material from soapstock and enzymes is 10:0.001 to 0.200, preferable 10:0.005.

The water amount in the soapstock is 10 to 60%, preferably lower than 40%

The distillator temperature in the wiped film evaporator is preferably in the range of 200-225° C., with a pressure of from 1 to 5 Torr.

Another subject of the invention is the use of fatty acid esters produced according to process of the invention as biodiesel.

### EXAMPLES

### Example 1.1

100 kg of soapstock with 40% of water, fatty part composed by 95.2% of fatty acids as soaps, 1.2% of monoglycerides, 1.5% of diglycerides, 1.1% of triglycerides, and 1.0% of sterols, measured by Gel permeation technique was neutralized with 8.7 kg of sulfuric acid 98% at 45C until PH 4.0. 9.0 kg of Methanol was added followed by 0.03 kg of liquid enzyme CALB—Candida antartica Lipase B from Novozymes. Mixture was kept under circulation using a diafragm pump 100 liter/hour flow rate during 6 days. Initial Acid value for the organic phase was 155 and decreased as described in the Table 1. External temperature ranged 24° C. to 30° C. over the 6 days.

TABLE 1
Acid value over 6 days

| days | AV mgkoh/g |
|---|---|
| 0 | 154 |
| 1 | 105 |
| 2 | 76 |
| 3 | 61 |
| 4 | 48 |
| 5 | 38 |
| 6 | 32 |

### Example 1.2

Circulation was stopped and mixture was settled out for 8 hours. About 49 kg of crude methyl esters was separated from 68.9 kg of an aqueous phase including a layer of a emulsion. The aqueous phase was filtered out through a press filter producing 11 kg of filtration cake. The filtered liquor was settled down for additional 3 hours separating 2.3 kg of crude methyl esters and 55.6 of a transparent aqueous phase which was discharged to sewer.

Total amount of crude methyl esters produced was 51.3 kg with AV 25. About 1.8 kg of sodium hydroxide 50% solution was added to neutralize the residual non esterified fatty acids.

Total amount after neutralization was 53.1 kg of neutralized crude esters for distillation.

### Example 1.3

The crude neutralized methyl esters was fed to a lab wiped film evaporator 0.13 ft2 at 1 kg/hour flow rate, still temperature was 220° C. working with 1.5 mmhg of pressure. A degasser 150C@5 mmhq was assembled before the main still to remove residual water coming from the neutralization and methanol.

Forecut of 40 kg of pure fatty acid methyl esters Gardner 4, AV<2 was produced as a main product. Process yield was 40% in relation to the soapstock and 71% in relation to the total fatty material.

11.1 kg of a bottom stream having 8% of sterols, partial glycerides, and fatty acid soaps was produced as residue. This material was processed according U.S. Pat. No. 6,281,373 B1 to recover the sterols and the fatty acids as methyl esters again. The bottom stream could also be recycled back to the soapstock storage tank.

### Example 2.1

100 kg of soapstock from the same source as example 1.1 was neutralized with 8.7 kg of sulfuric acid 98% at 45° C. until pH 4.0. 10.0 kg of Ethanol 96% was added followed by 0.03 kg of liquid enzyme CALB—Candida antartica Lipase B from Novozymes. Mixture was kept under circulation using a diafragm pump 80 liter/hour flow rate during 6 days. Initial Acid value for the organic phase was 150 and decreased to 38. External temperature ranged 22° C. to 32° C. over the 6 days.

### Example 2.2

Circulation was stopped and mixture was settled out for 6 hours. About 51 kg of crude methyl esters was separated from 68.9 kg of an aqueous phase including a very small layer of an emulsion. The aqueous phase was filtered out through a press filter producing 11 kg of filtration cake. The filtered liquor was settled down for additional 3 hours separating 1.3 kg of crude methyl esters and 55.2 of a transparent aqueous phase which was discharged to sewer. Total amount of crude ethyl esters produced was 52.0 kg with AV 38. About 3.0 kg of sodium hydroxide 50% solution was added to neutralize the residual non esterified fatty acids.

Total amount after neutralization was 55.0 kg of neutralized crude esters for distillation.

### Example 2.3

The crude neutralized ethyl esters was fed to a lab wiped film evaporator 0.13 ft2 at 1 kg/hour flow rate, still temperature was 230° C. working with 1.0 mmhg of pressure. A degasser 150C@5 mmhq was assembled before the main still to remove residual water coming from the neutralization and ethanol.

Forecut of 42 kg of pure fatty acid methyl esters Gardner 4, AV<2 was produced as a main product. Process yield was 42% in relation to the soapstock and 74% in relation to the total fatty material.

10.0 kg of a bottom stream having 8.4% of sterols, partial glycerides, and fatty acid soaps was produced as residue. This material was processed according US patent U.S. Pat. No. 6,281,373 B1 to recover the sterols. The bottom stream could also be recycled back to the soapstock storage tank.

### Example 3

1 kg of soapstock from the same source as example 1.1 was neutralized with 0.087 kg of sulfuric acid 98% at 45° C. until pH 4.0. 0.10 kg of Ethanol 96% was added followed by 0.08 kg of the same enzyme adsorbed on a macroporous resin—Novozym 435. The mixture was kept under slow mechanical agitation reaching AV 20 from initial AV of 155 after three hours of reaction. Temperature was 35° C. during the esterification time.

### Example 4

1 kg of soapstock from the same source as example 1.1 was neutralized with 0.087 kg of sulfuric acid 98% at 45C until PH 4.0. 0.09 kg of methanol was added followed by 0.0005 kg of liquid enzyme CALB—Candida Antartica Lipaze B from Novozymes. Mixture was kept under slow mechanical agitation Acid Value reached 18 from the initial 158 after 2 hours of reaction. Temperature was 30° C. during the reaction time.

We claim:

1. A process for producing fatty acid esters directly from a soapstock generated in an alkali refining process, which soapstock comprises 10-60% water, 0.1-2.0% of sterols, and 35-85% of fatty derivatives, including glycerides, by:
a) neutralizing and splitting the soaps with a strong acid until a pH of between 2 and 8 is reached;
b) enzymatically esterifying the soapstock using a lipase enzyme in a concentration ranging from 100 ppm to 10%, by weight, with a C₁-to-C₆ alkanol, in a weight ratio of 5-100%, in relation to the fatty components, at a temperature of from 15-to-70° C., in a batch or continuous process, to create a mixture, with a residual acid value, of crude esters and unesterified liquids and solids;
c) agitating the mixture mechanically or just by circulation during three-to-five days at temperatures of from 15-to-70° C., where the acid value of the fatty acids is measured in the organic layer during the days of agitation, and stopping the agitation when the acid value has not decreased further after 24 hours;
d) separating the ester phase from the mixture by settling, by a centrifuge, or by filtering out the solids, in order to facilitate separation of the components;
e) neutralizing the residual acid value from the incomplete enzymatic esterification of step b using an alkali solution selected from the group consisting of sodium hydroxide, potassium hydroxide, calcium hydroxide, aluminum hydroxide, lithium hydroxide, ammonium hydroxide, and derivatives of any of them;
f) distilling off the crude esters of step b using a batch technique or continuous flash distillators, where any residual amount of moisture from the soapstock or any aqueous phase added previously and/or methanol from step b is stripped off using a degasser just prior to this distillation to continuously produce 80 to 90% of light-colored esters.

2. The process according to claim 1, characterized in that the soapstock from alkali refining is selected from the group consisting of soybean, sunflower, rice, corn, coconut, palm kernel, rapeseed and cotton oil soapstocks.

3. The process according to claim 1, characterized in that the acid used to split the soaps is sulfuric acid or hydrochloridic acid, with a pH of from 3.5-to-6.

4. The process according to claim 1, characterized in that the lipase is produced by an organism selected from the group consisting of Aspergillus niger, Aspergillus oryzea, Bacillus species, Candida albicans, Candida antarctica, Candida cylindracea, Candida glabrata, Candida maltosa, Candida parapsilosis, Candida lipolytica, Candida tropicalis, Candida viswanathii, Chromobacterium viscosum, Geotrichum candidum, Issatchenkia orentalis, Kluyveromyces marxianus, Mucor javanicus, Penicilium camenberti, Penicilium roqueforti, Pichia guilliermondii, Porcine pancreas, Pseudomonas cepacia, Pseudomonas fluorescens, Rhizomucor miehei, Rhizopus arrhizus, Rhizopus oryzae, Rhizopus niveus, Rhizopus javanicus, Thermomyces lanugenosus, and mixtures thereof, each in the form of a liquid, a solid or immobilized in a substrate.

5. The process according to claim 4, characterized in that the lipase is a lipase of type B.

6. The process according to claim 5, characterized in that the lipase of type B is a Candida antarctica Lipase B.

7. The process according to claim 1, characterized in that the alkanol is a linear- or branched-C₁-to-C₆ alkanol.

8. The process according to claim 7, characterized in that the linear- or branched-C₁-to-C₆ alkanol is methanol or ethanol.

9. The process according to claim 1, characterized in that distillation step f is carried out by a batch or a continuous operation.

10. The process according to claim 9, characterized in that distillation step f is carried out in a thin-film or wiped-film evaporator.

11. The process according to claim 9, characterized in that distillation step f is carried out in a continuous distillation, operated at 180° C.-240° C. and 1-10 mm Hg pressure.

12. The process according to claim 11, characterized in that the continuous distillation is operated at 220° C. and 3 mm Hg pressure.

13. The process according to claim 1, characterized in that the distillation step f also produces a residue comprising 5-8% sterols.

14. Use of fatty acid esters produced according to claim 1 as biodiesel fuel.
